(19) United States
(12) Patent Application Publication
(10) Pub. No.: US 2023/0126248 A1
(43) Pub. Date: Apr. 27, 2023

(54) INTUBATION POSITIONING DEVICE

(71) Applicant: Michael KERNER, Cherry Hill, NJ (US)

(72) Inventor: Michael KERNER, Cherry Hill, NJ (US)

(21) Appl. No.: 17/974,315

(22) Filed: Oct. 26, 2022

Related U.S. Application Data
(60) Provisional application No. 63/272,124, filed on Oct. 26, 2021.

(51) Int. Cl. A61G 13/12

(52) U.S. Cl. CPC A61G 13/1215; A61G 13/1265; A61M 16/0488

(57) ABSTRACT

Technologies are disclosed for an intubation positioning device that may be configured to selectively adjust a head, neck, and/or an upper back of a patient lying on a surgical table. The device may comprise a body that may have a top, a bottom, a superior end relative to the neck, and/or an inferior end relative to the neck. The body may include an outer casing that permits expansion and contraction of the body in at least a vertical direction, perhaps via at least two inflatable chambers disposed in the body. At least a part of the superior end may be sized or shaped to receive the head of the patient. The device may include a controller configured to selectively permit entrance of a fluid into the first inflatable chamber and/or the second inflatable chamber that may be independently transitioned between an inflated shape and a deflated shape.

### CROSS-REFERENCE TO RELATED APPLICATIONS

The present application claims priority to and the benefit of U.S. Provisional Patent Application No. 63/272,124, filed Oct. 26, 2021, the contents of which are hereby incorporated in their entirety.

### TECHNICAL FIELD

The present disclosure is directed to an intubation positioning device and related methods.

### BACKGROUND

Intubation may include insertion of a tube into a patient's body, for example to provide a ventilation tube into the patient's trachea to facilitate breathing and/or to deliver breathing air or oxygen to the patient's pulmonary system. Intubation may be required to deliver a ventilation tube for relatively short time periods, such as for emergency treatment, or for surgical procedures. Intubation may also be used to deliver a ventilation tube for more long-term conditions, such as those that may require the patient to be serviced by a ventilator device. Patients may require an intubation procedure at various times, and for various reasons. For example, a patient may require a surgical procedure that may require anesthesia that could impair the patient's ability to breathe on their own. Also, the surgical procedure may target the patient's breathing airway, which may impair the patient's ability to breathe on their own. Otherwise, a patient might benefit from intubation due to some exigent medical condition, such as anaphylaxis, cardiac arrest, and/or head injury, among other conditions.

### SUMMARY

Technologies are disclosed for an intubation positioning device that may be configured to selectively adjust a head, neck, and upper back of a patient lying on a surgical table. The device may comprise a body. The body may include a top, a bottom, a superior end relative to the neck, and/or an inferior end relative to the neck. The inferior end may be spaced from the superior end along a longitudinal direction. The body may comprise at least two lateral sides that extend from the superior end to the inferior end along the longitudinal direction. The body may include an outer casing that may permit expansion and/or contraction of the body in at least a vertical direction. The vertical direction may be transverse to the longitudinal direction. At least a part of the superior end may be sized to receive the head of the patient, and/or may be shaped to receive the head of the patient.

The device may include a first inflatable chamber that may be disposed in the body. The first inflatable chamber may be substantially oriented along the longitudinal direction. A second inflatable chamber may be disposed in the body. The second inflatable chamber may be substantially oriented along the longitudinal direction. The second inflatable chamber may be disposed in a contiguous position relative to the first inflatable chamber.

In one or more embodiments, a first inflation port may be coupled to the first inflatable chamber. The first inflation port may be configured to permit a fluid to enter the first inflatable chamber. A second inflation port may be coupled to the second inflatable chamber. The second inflation port may be configured to permit the fluid to enter the second inflatable chamber.

In one or more embodiments, a controller may be coupled to the first and second inflation ports. The controller may be configured to (e.g., selectively) permit entrance of the fluid into the first inflatable chamber and/or the second inflatable chamber. The controller may (e.g., selectively) transition the first inflatable chamber and/or the second inflatable chamber (e.g., independently) between an inflated shape and a deflated shape.

In one or more embodiments, the device may comprise a third inflatable chamber that may be disposed in the body. The third inflatable chamber may be substantially oriented along the longitudinal direction. The third inflatable chamber may be disposed in a contiguous position relative to the second inflatable chamber.

In one or more embodiments, the device may comprise a third inflation port that may be coupled to the third inflatable chamber. The third inflation port may be configured to permit the fluid to enter the third inflatable chamber. The controller may be coupled to the third inflation port. The controller may be further configured to (e.g., selectively) permit entrance of the fluid into the third inflatable chamber. The controller may be configured to (e.g., selectively) transition the third inflatable chamber (e.g., independently) from the first inflatable chamber and/or the second inflatable chamber, between an inflated shape and a deflated shape.

In one or more embodiments, wherein the first inflatable chamber and the second inflatable chamber may be configured to (e.g., independently) transition between a fully-deflated shape, a partially inflated shape, and/or a fully-inflated shape.

In one or more embodiments, the superior end, the inferior end, and the at least two lateral sides may extend (e.g., substantially) perpendicular to the top and the bottom as the first inflatable chamber and/or the second inflatable chamber is transitioned from the fully-deflated shape to the fully-inflated shape.

In one or more embodiments, the outer casing of the body may be constructed of a flexible material.

In one or more embodiments, the first inflation port and/or the second inflation port may be one or more of a valve, a gauge cock, a plug, a tap, and/or a spigot.

In one or more embodiments, a fourth inflatable chamber may be disposed in the body. The fourth inflatable chamber may be (e.g., substantially) oriented along the longitudinal direction. The fourth inflatable chamber may be disposed in a contiguous position relative to the second inflatable chamber.

In one or more embodiments, a fifth inflatable chamber may be disposed in the body. The fifth inflatable chamber may be (e.g., substantially) oriented along the longitudinal direction. The fifth inflatable chamber may be disposed in a contiguous position relative to the fourth inflatable chamber.

In one or more embodiments, a sixth inflatable chamber may be disposed in the body. The sixth inflatable chamber may be (e.g., substantially) oriented along the longitudinal direction. The sixth inflatable chamber may be disposed in a contiguous position relative to the fourth inflatable chamber.

In one or more embodiments, a fourth inflation port may be coupled to the fourth inflatable chamber. The fourth inflation port may be configured to permit the fluid to enter the fourth inflatable chamber. The controller may be coupled to the fourth inflation port. The controller may be further configured to (e.g., selectively) permit entrance of the fluid into the fourth inflatable chamber. The controller may be further configured to (e.g., selectively) transition the fourth inflatable chamber (e.g., independently) from the first inflatable chamber, the second inflatable chamber, the third inflatable chamber, the fifth inflatable chamber, and/or the sixth inflatable chamber, between an inflated shape and a deflated shape.

In one or more embodiments, a fifth inflation port may be coupled to the fifth inflatable chamber. The fifth inflation port may be configured to permit the fluid to enter the fifth inflatable chamber. The controller may be coupled to the fifth inflation port. The controller may be further configured to (e.g., selectively) permit entrance of the fluid into the fifth inflatable chamber. The controller may be further configured to (e.g., selectively) transition the fifth inflatable chamber, independently from the first inflatable chamber, the second inflatable chamber, the third inflatable chamber, the fourth inflatable chamber, and/or the sixth inflatable chamber, between an inflated shape and a deflated shape.

In one or more embodiments, a sixth inflation port may be coupled to the sixth inflatable chamber. The sixth inflation port may be configured to permit the fluid to enter the sixth inflatable chamber. The controller may be coupled to the sixth inflation port. The controller may be further configured to (e.g., selectively) permit entrance of the fluid into the sixth inflatable chamber. The controller may be configured to (e.g., selectively) transition the sixth inflatable chamber (e.g., independently) from the first inflatable chamber, the second inflatable chamber, the third inflatable chamber, the fourth inflatable chamber, and/or the fifth inflatable chamber, between an inflated shape and a deflated shape.

In one or more embodiments, the third inflatable chamber, the fourth inflatable chamber, the fifth inflatable chamber, and/or the sixth inflatable chamber may be configured to (e.g., independently) transition between a fully-deflated shape, a partially inflated shape, and/or a fully-inflated shape.

In one or more embodiments, the first inflatable chamber and/or the second inflatable chamber may define at least a portion of the outer casing. In one or more embodiments, the outer casing may be formed (e.g., entirely, or at least a portion) by the exteriors of the inflatable chambers of the intubation positioning device. In one or more embodiments, the outer casing may be a physically separate/removable layer from the one or more inflatable chambers of the intubation positioning device.

In one or more embodiments, the body may have a length that may extend along the longitudinal direction from the superior end to the inferior end. The body may have a width that may extend from a first lateral side of the at least two lateral sides to a second lateral side of the at least two lateral sides along a transverse direction that may be perpendicular to the longitudinal direction and/or the vertical direction. The length may be greater than the width, perhaps for example when the body may be positioned on the surgical table in preparation to receive the patient.

In one or more embodiments, the body may have a length that may extend along the longitudinal direction from the superior end to the inferior end. The body may have a width that may extend from a first lateral side of the at least two lateral sides to a second lateral side of the at least two lateral sides along a transverse direction that may be perpendicular to the longitudinal direction and/or the vertical direction. The width may be greater than the length, perhaps for example when the body may be positioned on the surgical table in preparation to receive the patient.

In one or more embodiments, the superior end may define a first height that may extend from the bottom to the top along the vertical direction. The inferior end may define a second height that may extend from the bottom to the top along a vertical direction. The first height may be greater than the second height upon at least a partial inflation of the first inflatable chamber and/or the second inflatable chamber.

In one or more embodiments, the body may be configured such that the first inflatable chamber may be disposed (e.g., substantially) contiguous to the second inflatable chamber in a position above the second inflatable chamber relative to the vertical direction, or in a position beneath the second inflatable chamber relative to the vertical direction. The first inflatable chamber may be disposed closer to the superior end relative to the third inflatable chamber.

The third inflatable chamber may be disposed (e.g., substantially) contiguous to the fourth inflatable chamber in a position above the fourth inflatable chamber relative to the vertical direction, or in a position beneath the fourth inflatable chamber relative to the vertical direction. The fourth inflatable chamber may be disposed relative to the second inflatable chamber in a (e.g., substantially) lateral contiguous position, or a (e.g., substantially) diagonal contiguous position.

The fifth inflatable chamber may be disposed substantially contiguous to the sixth inflatable chamber in a position above the sixth inflatable chamber relative to the vertical direction, or in a position beneath the sixth inflatable chamber relative to the vertical direction. The sixth inflatable chamber may be disposed relative to the third inflatable chamber in a (e.g., substantially) lateral contiguous position, or in a (e.g., substantially) diagonal contiguous position. The sixth inflatable chamber may be disposed closer to the inferior end relative to the third inflatable chamber.

In one or more embodiments, the body may be configured such that the first inflatable chamber may be disposed in a (e.g., substantially) lateral contiguous position relative to the second inflatable chamber, the fourth inflatable chamber, and/or the fifth inflatable chamber. The first inflatable chamber may be disposed closer to the superior end relative to the fourth inflatable chamber.

The second inflatable chamber may be disposed in a (e.g., substantially) lateral contiguous position relative to the first inflatable chamber, the third inflatable chamber, the fourth inflatable chamber, and/or the fifth inflatable chamber. The fifth inflatable chamber may be disposed in a (e.g., substantially) lateral contiguous position relative to the second inflatable chamber, the third inflatable chamber, the fourth inflatable chamber, and/or the sixth inflatable chamber.

The sixth inflatable chamber may be disposed in a (e.g., substantially) lateral contiguous position relative to the fourth inflatable chamber, and/or the fifth inflatable chamber. The sixth inflatable chamber may be disposed closer to the inferior end relative to the fourth inflatable chamber.

In one or more embodiments, the body may be configured such that the first inflatable chamber may be disposed (e.g., substantially) contiguous to the second inflatable chamber in a position above the second inflatable chamber relative to the vertical direction, or a position beneath the second inflatable chamber relative to the vertical direction. The first inflatable chamber may be disposed closer to the superior end relative to the third inflatable chamber. The third inflatable chamber may be disposed (e.g., substantially) contiguous to the first inflatable chamber, and/or the second inflatable chamber. The third inflatable chamber may be disposed closer to the inferior end relative to the first inflatable chamber.

In one or more embodiments, the body may be configured such that the first inflatable chamber may be disposed in a (e.g., substantially) lateral contiguous position relative to the second inflatable chamber. The third inflatable chamber may be disposed (e.g., substantially) contiguous to the first inflatable chamber, and/or the second inflatable chamber. The third inflatable chamber may be disposed closer to the inferior end relative to the first inflatable chamber.

In one or more embodiments, the body may be configured such that the first inflatable chamber may be disposed (e.g., substantially) contiguous to the second inflatable chamber in a position above the second inflatable chamber relative to the vertical direction, or a position beneath the second inflatable chamber relative to the vertical direction. The first inflatable chamber may be disposed in a position closer to the bottom relative to the second inflatable chamber, or a position closer to the top relative to the second inflatable chamber.

In one or more embodiments, the body may be configured such that the first inflatable chamber may be disposed in a (e.g., substantially) lateral contiguous position relative to the second inflatable chamber. The second inflatable chamber may be disposed in a position closer to the inferior end relative to the first inflatable chamber, or in a position closer to the superior end relative to the first inflatable chamber.

In one or more embodiments, one or more techniques are disclosed for positioning at least a head of a patient lying on a surgical table. One or more techniques may include positioning an intubation device beneath at least the head of the patient lying on the surgical table. The intubation device may include a body. The body may have a superior end, an inferior end, a bottom, and/or a top. The superior end may be configured to position at least the head of the patient along the top of the intubation device such that, for example, the inferior end may be proximate to a neck of the patient, or an upper back of the patient.

One or more techniques may include causing a first inflatable chamber and/or a second inflatable chamber disposed in the body to (e.g., independently) expand until a spine of the patient may be aligned with an orientation of the head and the neck of the patient.

One or more techniques may include causing at least a third inflatable chamber disposed in the body to (e.g., independently) expand until the spine of the patient may be aligned with an orientation of the head and the neck of the patient.

One or more techniques may include causing a fourth inflatable chamber and/or a fifth inflatable chamber disposed in the body to (e.g., independently) expand until a cervical spine of the patient is aligned with an orientation of the head and the neck of the patient.

One or more techniques may include causing at least a sixth inflatable chamber disposed in the body to (e.g., independently) expand until a thoracic spine of the patient may be aligned with an orientation of the head and the neck of the patient.

### BRIEF DESCRIPTION OF DRAWINGS

The elements and other features, advantages and disclosures contained herein, and the manner of attaining them, will become apparent and the present disclosure will be better understood by reference to the following description of various examples of the present disclosure taken in conjunction with the accompanying drawings.

FIG. 1 illustrates an example of an inflatable multi-chamber intubation positioning device;

FIG. 2 illustrates an example schematic of one or more inflatable chambers of an inflatable multi-chamber intubation positioning device;

FIG. 3 illustrates an example schematic of one or more inflatable chambers of an inflatable multi-chamber intubation positioning device;

FIG. 4 is a block diagram of a hardware configuration of an example control processor;

FIG. 5 illustrates an example schematic of one or more inflatable chambers of an inflatable multi-chamber intubation positioning device;

FIG. 6 illustrates an example schematic of one or more inflatable chambers of an inflatable multi-chamber intubation positioning device;

FIG. 7 illustrates an example schematic of one or more inflatable chambers of an inflatable multi-chamber intubation positioning device;

FIG. 8 illustrates an example schematic of one or more inflatable chambers of an inflatable multi-chamber intubation positioning device;

FIG. 9 illustrates an example schematic of one or more inflatable chambers of an inflatable multi-chamber intubation positioning device;

FIG. 10 illustrates an example schematic of one or more inflatable chambers of an inflatable multi-chamber intubation positioning device;

FIG. 11 illustrates an example schematic of one or more inflatable chambers of an inflatable multi-chamber intubation positioning device; and

FIG. 12 is a diagram of an example technique for positioning a patient using an intubation positioning device.

### DETAILED DESCRIPTION

Intubation is a skill practiced by trained medical personnel as successful intubation is executed by avoiding conflicts with the patient's teeth, mouth, tongue, esophagus, and/or larynx, among other potential conflicts or difficulties. For many intubations, the patient may be sedated or unconscious. The patient may be flat on their back, such as on a surgical table or in a position where an injury or a sudden illness occurred. The practitioner performing the intubation (e.g., doctor, nurse, nurse practitioner, paramedic, etc.) may open the patient's mouth and—relying on training and experience to avoid potential conflicts with the patient's teeth, lower jaw, esophagus, etc.—insert/guide the intubation tube down the patient's throat into the patient's airway/trachea. A (e.g., small) balloon around the intubation tube may be inflated to hold the intubation tube in place and/or to control the flow of air (e.g., through the intubation tube and not past or around the intubation tube). The tube may be secured in the airway and/or may be taped in place at the mouth.

One or more devices and/or techniques described herein may provide the practitioner with means to avoid one or more difficulties encountered in the intubation process. For example, a particular alignment of the patient's head, neck (e.g., cervical spine), and/or upper back (e.g., thoracic spine) may facilitate successful intubation. Stated somewhat differently, devices and techniques described herein may provide for proper alignment of the patient's head, neck/throat/cervical spine, and/or upper back/thoracic spine. Such alignment capabilities may facilitate successful and/or efficient intubation, for example, as compared to situations in which the practitioner cannot arrange for a desired/controlled/optimum alignment of the aforementioned sections of the patient's anatomy.

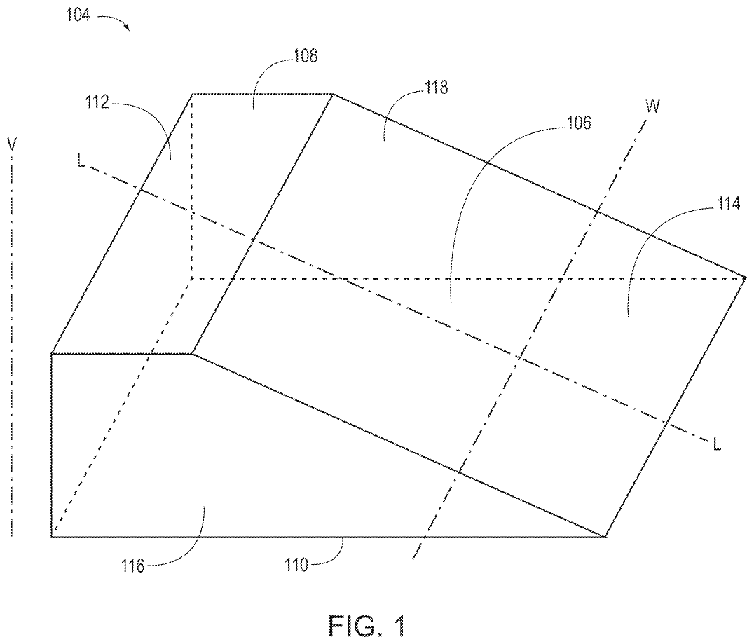

FIG. 1 illustrates an example of an inflatable multi-chamber intubation positioning device 104. Referring to FIG. 1, technologies and techniques are described herein for intubation positioning device 104 that may be configured to selectively adjust a head, neck, and upper back of a patient (not shown) lying on a surgical table (not shown). The device 104 may comprise a body 106. The body 106 may include a top 108, a bottom 110, a superior end 112 relative to the neck, and/or an inferior end 114 relative to the neck. The inferior end 114 may be spaced from the superior end 112 along a longitudinal direction L. The body 106 may comprise at least two lateral sides 116 and 118 that extend from the superior end 112 to the inferior end 114 along the longitudinal direction L. The body 106 may include an outer casing (not shown) that may permit expansion and/or contraction of the body 106 in at least a vertical direction V. The vertical direction V may be transverse to the longitudinal direction L. At least a part of the superior end 112 may be sized to receive the head of the patient, and/or may be shaped (not shown) to receive the head of the patient.

In one or more embodiments, the body 106 may, perhaps for example upon full expansion, extend along the longitudinal direction L around 20 to 36 inches, for example. The body, perhaps for example upon full expansion, extend along a width direction W (e.g., perpendicular to the longitudinal direction L) around 20 to 36 inches, for example.

In one or more embodiments, the body 106 may have a length that may extend along the longitudinal direction L from the superior end 112 to the inferior end 114. The body 106 may have a width that may extend from a first lateral side 116 of the at least two lateral sides to a second lateral side 118 of the at least two lateral sides along a transverse direction that may be perpendicular to the longitudinal direction L and/or the vertical direction V. The length may be greater than the width, perhaps for example when the body 106 may be positioned on the surgical table in preparation to receive the patient (not shown).

In one or more embodiments, the body 106 may have a length that may extend along the longitudinal direction L from the superior end 112 to the inferior end 114. The body 106 may have a width that may extend from the first lateral side 116 of the at least two lateral sides to the second lateral side 118 of the at least two lateral sides along a transverse direction that may be perpendicular to the longitudinal direction L and/or the vertical direction V. The width may be greater than the length, perhaps for example when the body 106 may be positioned on the surgical table in preparation to receive the patient (not shown).

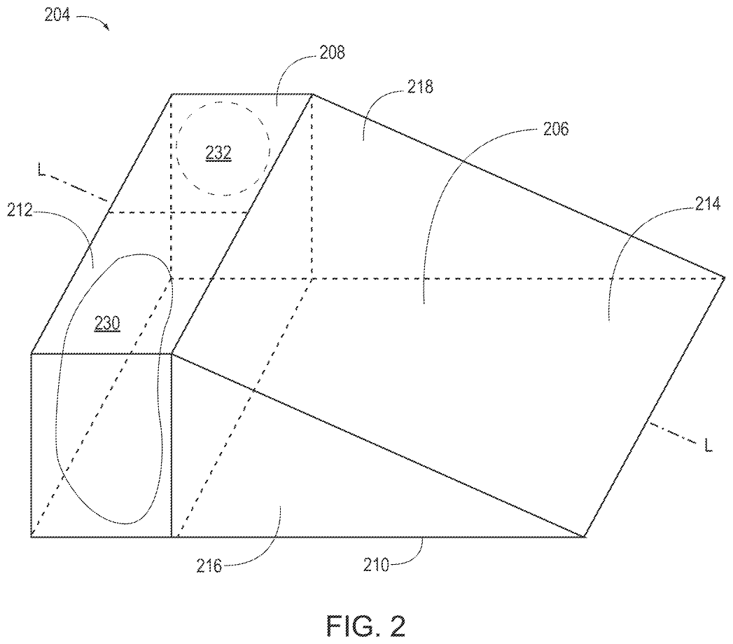

FIG. 2 illustrates an example schematic of one or more inflatable chambers of an inflatable multi-chamber intubation positioning device 204. The device 204 may include a first inflatable chamber 230 that may be disposed in the body 206. The first inflatable chamber 230 may be substantially oriented along the longitudinal direction L. A second inflatable chamber 232 may be disposed in the body 206. The second inflatable chamber 232 may be substantially oriented along the longitudinal direction L. The second inflatable chamber 232 may be disposed in a contiguous position relative to the first inflatable chamber 230.

In one or more embodiments, a first inflation port (not shown) may be coupled to the first inflatable chamber 230. The first inflation port may be configured to permit a fluid (e.g., air, water, nitrogen, etc.) to enter the first inflatable chamber 230. A second inflation port (not shown) may be coupled to the second inflatable chamber 232. The second inflation port may be configured to permit the fluid (e.g., air, water, nitrogen, etc.) to enter the second inflatable chamber 232.

In one or more embodiments, a controller (not shown) may be coupled to the first and/or second inflation ports. The controller may be configured to (e.g., selectively) permit entrance of the fluid into the first inflatable chamber 230 and/or the second inflatable chamber 232. The controller may (e.g., selectively) transition the first inflatable chamber 230 and/or the second inflatable chamber 232 (e.g., independently) between an inflated shape and a deflated shape (not shown).

In one or more embodiments, the superior end 212, the inferior end 214, and the at least two lateral sides 216 and 218 may extend (e.g., substantially) perpendicular to the top 208 and the bottom 210 as the first inflatable chamber 230 and/or the second inflatable chamber 232 is transitioned from the fully-deflated shape to the fully-inflated shape (not shown).

In one or more embodiments, the superior end 212 may define a first height (not shown) that may extend from the bottom 210 to the top 208 along the vertical direction V. The inferior end 214 may define a second height (not shown) that may extend from the bottom 210 to the top 208 along a vertical direction V. The first height may be greater than the second height upon at least a partial inflation of the first inflatable chamber 230 and/or the second inflatable chamber 232.

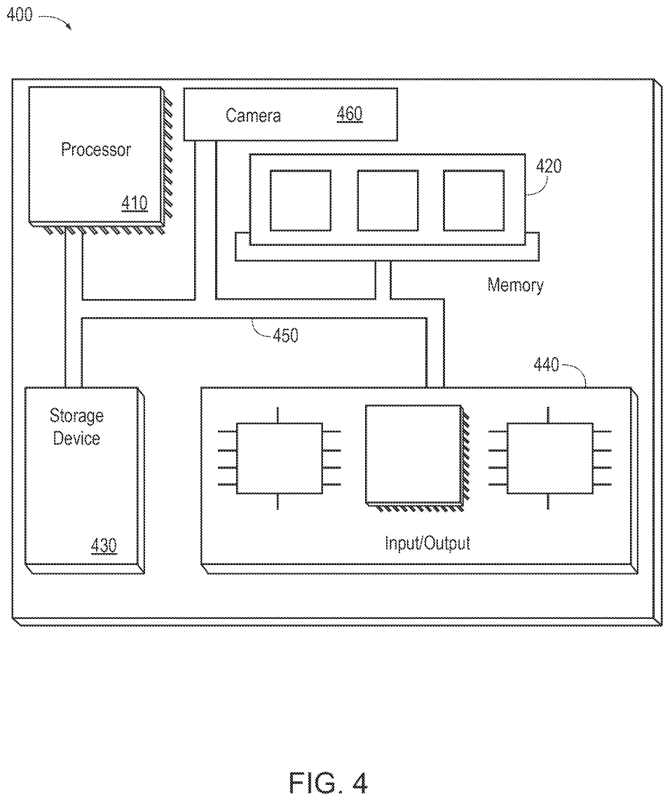

FIG. 4 is a block diagram of a hardware configuration of an example control processor (e.g., “processor”, “control module”, etc.) that may be used by a controller (not shown) to control the inflation and/or deflation of one or more inflatable chambers described herein. The hardware configuration 400 may be operable to facilitate delivery of information from an internal server of a device. The hardware configuration 400 can include a processor 410, a memory 420, a storage device 430, and/or an input/output device 440. One or more of the components 410, 420, 430, and 440 can, for example, be interconnected using a system bus 450. The processor 410 can process instructions for execution within the hardware configuration 400. The processor 410 can be a single-threaded processor or the processor 410 can be a multi-threaded processor. The processor 410 can be capable of processing instructions stored in the memory 420 and/or on the storage device 430.

The memory 420 can store information within the hardware configuration 400. The memory 420 can be a computer-readable medium (CRM), for example, a non-transitory CRM. The memory 420 can be a volatile memory unit, and/or can be a non-volatile memory unit.

The storage device 430 can be capable of providing mass storage for the hardware configuration 400. The storage device 430 can be a computer-readable medium (CRM), for example, a non-transitory CRM. The storage device 430 can, for example, include a hard disk device, an optical disk device, flash memory and/or some other large capacity storage device. The storage device 430 can be a device external to the hardware configuration 400.

The input/output device 440 may provide input/output operations for the hardware configuration 400. The input/output device 440 (e.g., a transceiver device) can include one or more of a network interface device (e.g., an Ethernet card), a serial communication device (e.g., an RS-232 port), one or more universal serial bus (USB) interfaces (e.g., a USB 2.0 port) and/or a wireless interface device (e.g., an 802.11 card). The input/output device can include driver devices configured to send communications to, and/or receive communications from one or more networks (not shown).

A camera device 460 may provide digital video input/output capability for the hardware configuration 400. The camera device 460 may communicate with any of the elements of the hardware configuration 400, perhaps for example via system bus 450. The camera device 460 may capture digital images and/or may scan images of various kinds, such as Universal Product Code (UPC) codes and/or Quick Response (QR) codes, for example, among other images.

The camera device 460 may include at least one microphone device and/or at least one speaker device. The input/output of the camera device 460 may include audio signals/packets/components, perhaps for example separate/separable from, or in some (e.g., separable) combination with, the video signals/packets/components of the camera device 460.

The camera device 460 may be in wired and/or wireless communication with the hardware configuration 400. In one or more embodiments, the camera device 460 may be external to the hardware configuration 400. In one or more embodiments, the camera device 460 may be internal to the hardware configuration 400.

In one or more embodiments, technologies that may provide for capabilities to align a patient's head, neck (e.g., cervical spine), and/or upper back (e.g., thoracic spine) to facilitate the patient's intubation, among other embodiments, could be useful. Further, techniques that may provide for more successful patient intubation by aligning a patient's head, neck (e.g., cervical spine), and/or upper back (e.g., thoracic spine) to a desired/optimum orientation could be useful.

In one or more embodiments, the controller may monitor/measure the pressure in, and/or fluid flow to/from (e.g., via the ports), one or more of the inflatable chambers disposed in the body (e.g., individually and/or in configurable groups of two or more, etc.) of the intubation positioning devices described herein. The controller may provide for electronic control of over-pressurization of one or more of the inflatable chambers. For example, the controller may lower the pressure in an inflatable chamber (e.g., by deflating the inflatable chamber), perhaps for example to lower the potential of a failure of the inflatable chamber (e.g., avoid and/or lower the potential of the inflatable chamber bursting).

In one or more embodiments, the controller may control the pressure in, and/or fluid flow to/from (e.g., via the ports), one or more of the inflatable chambers that may be disposed in the body (e.g., individually and/or in configurable groups of two or more, etc.). The pressure and/or fluid flow control may provide the motivation/source/energy to transfer one or more of the inflatable chambers (e.g., individually and/or in configurable groups of two or more, etc.) to transition the one or more inflatable chambers between and/or among a fully-deflated shape/state/condition, a partially-inflatable shape/state/condition, and/or a fully-inflatable shape/state/condition.

In one or more embodiments, the controller may be configurable (e.g., via a user interface—not shown) to accept a user input (e.g., from an intubation practitioner) to change the inflation state of one or more of the inflatable chambers. The controller may change the inflation state of the one or more inflatable chambers individually, perhaps for example as selected by the practitioner. The controller may change the inflation state of the one or more inflatable chambers, perhaps as a group of two or more inflatable chambers. Groups of one or more inflatable chambers may be configured by the user, perhaps for example by the practitioner via the user interface. Particular inflation states (e.g., inflation positions) may be configured by the user, perhaps for example via the user interface. For example, a practitioner may select from one or more preconfigured inflation positions for an intubation positioning device. For example, at least one preconfigured inflation position may cause the transition of one or more of the inflatable chambers in the device to (e.g., individually) preconfigured inflation states. In one or more embodiments, a practitioner may select a preconfigured inflation state based on the patient's weight, height, frame size, and/or neck size, etc., among other patient characteristics, for example.

Without the capabilities, techniques, methods, and/or devices described herein, the skilled artisan would not appreciate how to efficiently and/or effectively provide for desired/optimum alignment of a patient's head, neck, and/or upper back such that patient intubation may be more successful. Such desired/optimum alignment may facilitate avoiding intubation conflicts with the patient's teeth, lower jaw, and/or esophagus, among other potential conflicts with intubation.

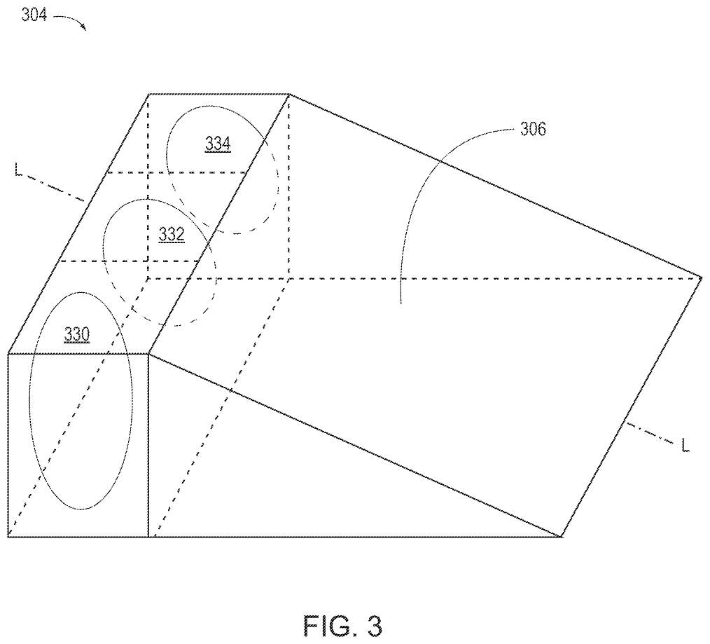

FIG. 3 illustrates an example schematic of one or more inflatable chambers of an inflatable multi-chamber intubation positioning device 304. In one or more embodiments, the device 304 may comprise a first inflatable chamber 330, a second inflatable chamber 332, and/or a third inflatable chamber 334 that may be disposed in the body 306. The third inflatable chamber 334 may be substantially oriented along the longitudinal direction L. The third inflatable chamber 334 may be disposed in a contiguous position relative to the second inflatable chamber 332.

In one or more embodiments, the device 304 may comprise a third inflation port (not shown) that may be coupled to the third inflatable chamber. The third inflation port may be configured to permit the fluid (e.g., air, water, nitrogen, etc.) to enter the third inflatable chamber 334. The controller may be coupled to the third inflation port. The controller may be further configured to (e.g., selectively) permit entrance of the fluid into the third inflatable chamber 334. The controller may be configured to (e.g., selectively) transition the third inflatable chamber 334 (e.g., independently) from the first inflatable chamber 330 and/or the second inflatable chamber 332, between an inflated shape and a deflated shape (not shown).

In one or more embodiments, the first inflatable chamber 330, the second inflatable chamber 332, and/or the third inflatable chamber 334 may be configured to (e.g., independently) transition between a fully-deflated shape, a partially inflated shape, and/or a fully-inflated shape (not shown).

In one or more embodiments, the outer casing of the body 306 may be constructed of a flexible material. Exemplary material may include coated textile materials, polymeric membranes, laminates, and the like. In one or more embodiments, the first inflatable chamber 330, the second inflatable chamber 332, and/or the third inflatable chamber 334 may be constructed separately and/or combined together (e.g., with adhesive, cement, bindings, etc.) and/or with other parts/elements of the body 306. In one or more embodiments, the first inflatable chamber 330, the second inflatable chamber 332, and/or the third inflatable chamber 334 may be bored/carved/formed out of a substrate of/from which the body 306 may be constructed. In one or more embodiments, the first inflatable chamber 330, the second inflatable chamber 332, and/or the third inflatable chamber 334 may define at least a portion of the outer casing of the body 306.

In one or more embodiments, the first inflation port, the second inflation port, and/or the third inflation port may be one or more of a valve, a gauge cock, a plug, a tap, and/or a spigot.

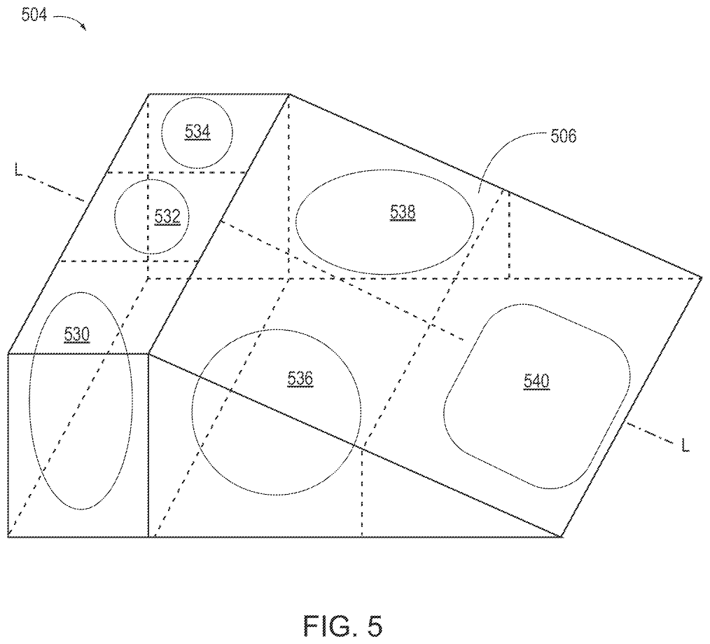

FIG. 5 illustrates an example schematic of one or more inflatable chambers of an inflatable multi-chamber intubation positioning device 504. In one or more embodiments, a fourth inflatable chamber 536 may be disposed in the body 506. The fourth inflatable chamber 536 may be (e.g., substantially) oriented along the longitudinal direction L. The fourth inflatable chamber 536 may be disposed in a contiguous position relative to the second inflatable chamber 532.

In one or more embodiments, a fifth inflatable chamber 538 may be disposed in the body 506. The fifth inflatable chamber 538 may be (e.g., substantially) oriented along the longitudinal direction L. The fifth inflatable chamber 538 may be disposed in a contiguous position relative to the fourth inflatable chamber 536.

In one or more embodiments, a sixth inflatable chamber 540 may be disposed in the body 506. The sixth inflatable chamber 540 may be (e.g., substantially) oriented along the longitudinal direction L. The sixth inflatable chamber 540 may be disposed in a contiguous position relative to the fourth inflatable chamber 536.

In one or more embodiments, a fourth inflation port (not shown) may be coupled to the fourth inflatable chamber 536. The fourth inflation port may be configured to permit the fluid (e.g., air, water, nitrogen, etc.) to enter the fourth inflatable chamber 536. The controller may be coupled to the fourth inflation port. The controller may be further configured to (e.g., selectively) permit entrance of the fluid into the fourth inflatable chamber 536. The controller may be further configured to (e.g., selectively) transition the fourth inflatable chamber 536 (e.g., independently) from the first inflatable chamber 530, the second inflatable chamber 532, the third inflatable chamber 534, the fifth inflatable chamber 538, and/or the sixth inflatable chamber 540, between an inflated shape and a deflated shape (not shown).

In one or more embodiments, a fifth inflation port (not shown) may be coupled to the fifth inflatable chamber 538. The fifth inflation port may be configured to permit the fluid to enter the fifth inflatable chamber 538. The controller may be coupled to the fifth inflation port. The controller may be further configured to (e.g., selectively) permit entrance of the fluid into the fifth inflatable chamber 538. The controller may be further configured to (e.g., selectively) transition the fifth inflatable chamber 538, independently from the first inflatable chamber 530, the second inflatable chamber 532, the third inflatable chamber 534, the fourth inflatable chamber 536, and/or the sixth inflatable chamber 540, between an inflated shape and a deflated shape (not shown).

In one or more embodiments, a sixth inflation port (not shown) may be coupled to the sixth inflatable chamber 540. The sixth inflation port may be configured to permit the fluid to enter the sixth inflatable chamber 540. The controller may be coupled to the sixth inflation port. The controller may be further configured to (e.g., selectively) permit entrance of the fluid into the sixth inflatable chamber 540. The controller may be configured to (e.g., selectively) transition the sixth inflatable chamber 540 (e.g., independently) from the first inflatable chamber 530, the second inflatable chamber 532, the third inflatable chamber 534, the fourth inflatable chamber 536, and/or the fifth inflatable chamber 538, between an inflated shape and a deflated shape (not shown).

In one or more embodiments, the first inflatable chamber 530, the second inflatable chamber 532, the third inflatable chamber 534, the fourth inflatable chamber 536, the fifth inflatable chamber 538, and/or the sixth inflatable chamber 540 may be configured to (e.g., independently) transition between a fully-deflated shape, a partially inflated shape, and/or a fully-inflated shape.

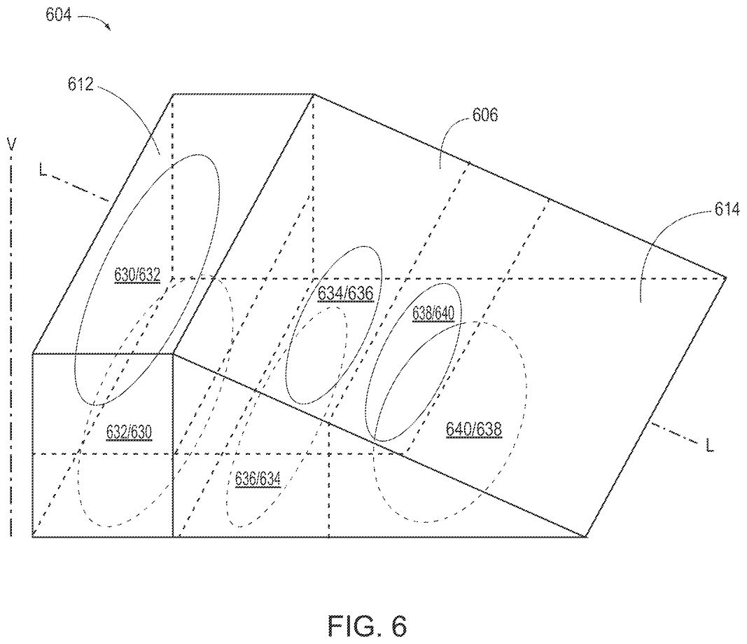

FIG. 6 illustrates an example schematic of one or more inflatable chambers of an inflatable multi-chamber intubation positioning device 604. In one or more embodiments, the body 606 may be configured such that the first inflatable chamber 630 may be disposed (e.g., substantially) contiguous to the second inflatable chamber 632 in a position above the second inflatable chamber 632 relative to the vertical direction V, or in a position beneath the second inflatable chamber 632 relative to the vertical direction V. The first inflatable chamber 630 may be disposed closer to the superior end 612 relative to the third inflatable chamber 634.

The third inflatable chamber 634 may be disposed (e.g., substantially) contiguous to the fourth inflatable chamber 636 in a position above the fourth inflatable chamber 636 relative to the vertical direction V, or in a position beneath the fourth inflatable chamber 636 relative to the vertical direction V. The fourth inflatable chamber 636 may be disposed relative to the second inflatable chamber 632 in a (e.g., substantially) lateral contiguous position, or a (e.g., substantially) diagonal contiguous position.

The fifth inflatable chamber 638 may be disposed substantially contiguous to the sixth inflatable chamber 640 in a position above the sixth inflatable chamber 640 relative to the vertical direction V, or in a position beneath the sixth inflatable chamber 640 relative to the vertical direction V. The sixth inflatable chamber 640 may be disposed relative to the third inflatable chamber 634 in a (e.g., substantially) lateral contiguous position, or in a (e.g., substantially) diagonal contiguous position. The sixth inflatable chamber 640 may be disposed closer to the inferior end 614 relative to the third inflatable chamber 634.

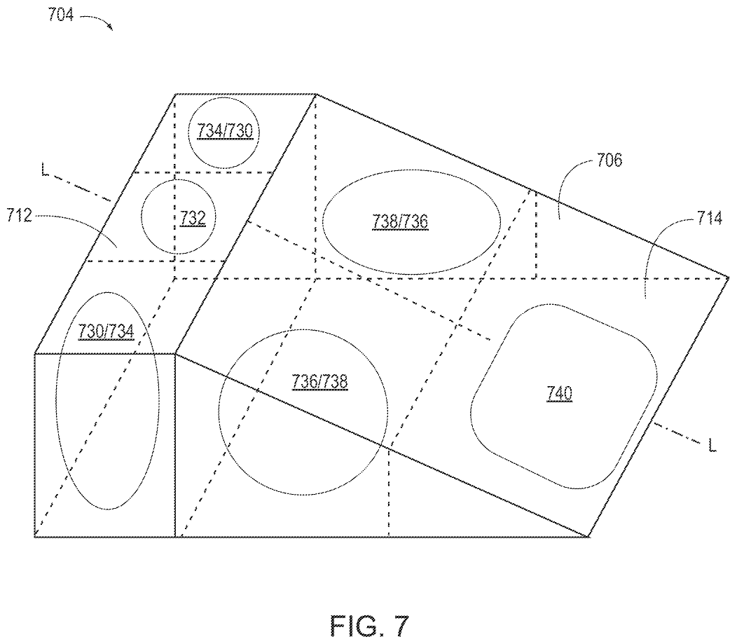

FIG. 7 illustrates an example schematic of one or more inflatable chambers of an inflatable multi-chamber intubation positioning device 704. In one or more embodiments, the body 706 may be configured such that the first inflatable chamber 730 may be disposed in a (e.g., substantially) lateral contiguous position relative to the second inflatable chamber 732, the fourth inflatable chamber 736, and/or the fifth inflatable chamber 738. The first inflatable chamber 730 may be disposed closer to the superior end 712 relative to the fourth inflatable chamber 736.

The second inflatable chamber 732 may be disposed in a (e.g., substantially) lateral contiguous position relative to the first inflatable chamber 730, the third inflatable chamber 734, the fourth inflatable chamber 736, and/or the fifth inflatable chamber 738. The fifth inflatable chamber 738 may be disposed in a (e.g., substantially) lateral contiguous position relative to the second inflatable chamber 732, the third inflatable chamber 734, the fourth inflatable chamber 736, and/or the sixth inflatable chamber 740.

The sixth inflatable chamber 740 may be disposed in a (e.g., substantially) lateral contiguous position relative to the fourth inflatable chamber 736, and/or the fifth inflatable chamber 738. The sixth inflatable chamber 740 may be disposed closer to the inferior end 714 relative to the fourth inflatable chamber 736.

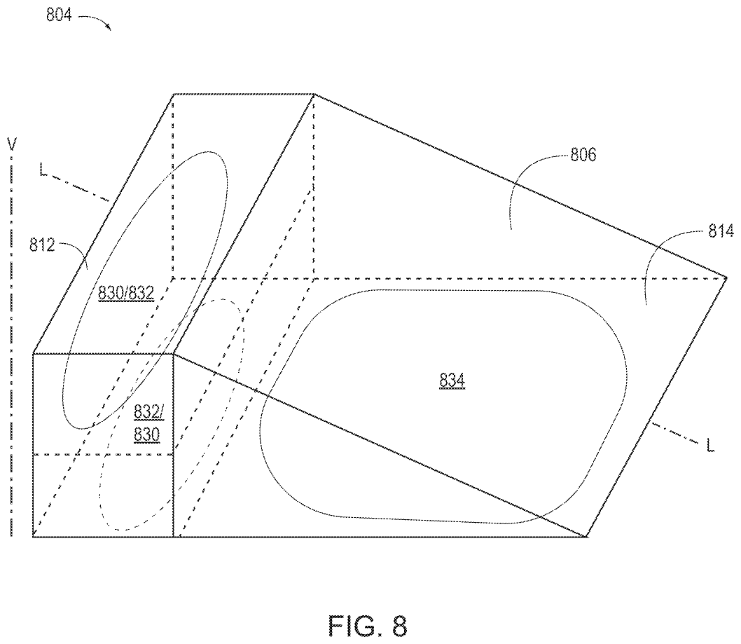

FIG. 8 illustrates an example schematic of one or more inflatable chambers of an inflatable multi-chamber intubation positioning device 804. In one or more embodiments, the body 806 may be configured such that the first inflatable chamber 830 may be disposed (e.g., substantially) contiguous to the second inflatable chamber 832 in a position above the second inflatable chamber 832 relative to the vertical direction V, or a position beneath the second inflatable chamber 832 relative to the vertical direction V. The first inflatable chamber 830 may be disposed closer to the superior end 812 relative to the third inflatable chamber 834. The third inflatable chamber 834 may be disposed (e.g., substantially) contiguous to the first inflatable chamber 830, and/or the second inflatable chamber 832. The third inflatable chamber 834 may be disposed closer to the inferior end 814 relative to the first inflatable chamber 830.

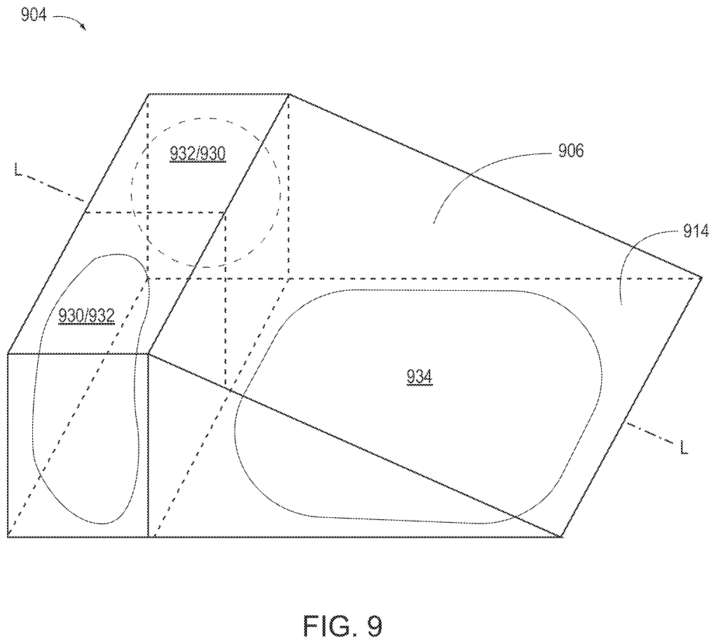

FIG. 9 illustrates an example schematic of one or more inflatable chambers of an inflatable multi-chamber intubation positioning device 904. In one or more embodiments, the body 906 may be configured such that the first inflatable chamber 930 may be disposed in a (e.g., substantially) lateral contiguous position relative to the second inflatable chamber 932. The third inflatable chamber 934 may be disposed (e.g., substantially) contiguous to the first inflatable chamber 930, and/or the second inflatable chamber 932. The third inflatable chamber 934 may be disposed closer to the inferior end 914 relative to the first inflatable chamber 930.

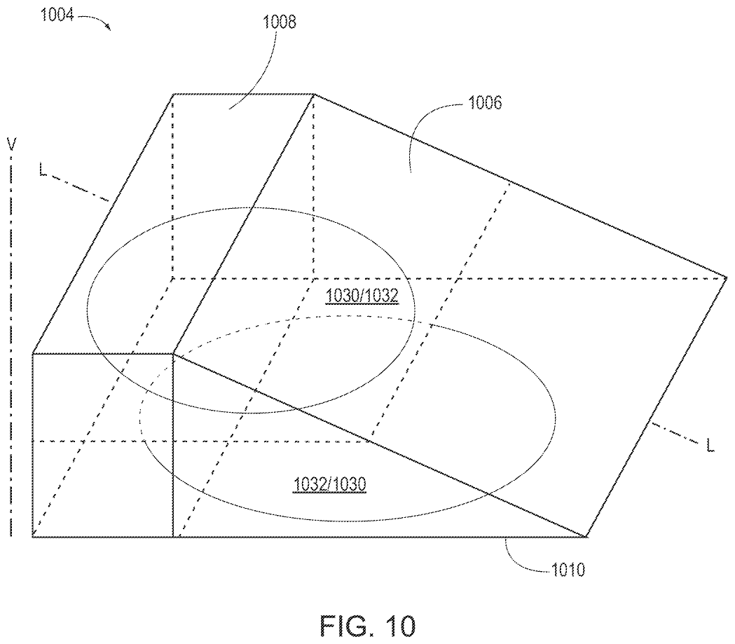

FIG. 10 illustrates an example schematic of one or more inflatable chambers of an inflatable multi-chamber intubation positioning device 1004. In one or more embodiments, the body 1006 may be configured such that the first inflatable chamber 1030 may be disposed (e.g., substantially) contiguous to the second inflatable chamber 1032 in a position above the second inflatable chamber 1032 relative to the vertical direction V, or a position beneath the second inflatable chamber 1032 relative to the vertical direction V. The first inflatable chamber 1030 may be disposed in a position closer to the bottom 1010 relative to the second inflatable chamber 1032, or a position closer to the top 1008 relative to the second inflatable chamber 1032.

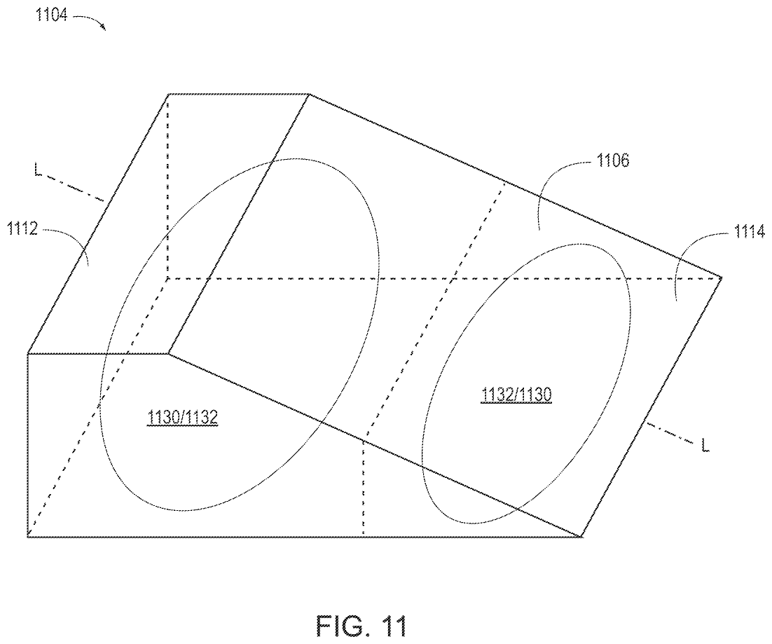

FIG. 11 illustrates an example schematic of one or more inflatable chambers of an inflatable multi-chamber intubation positioning device 1104. In one or more embodiments, the body 1106 may be configured such that the first inflatable chamber 1130 may be disposed in a (e.g., substantially) lateral contiguous position relative to the second inflatable chamber 1132. The second inflatable chamber 1132 may be disposed in a position closer to the inferior end 1114 relative to the first inflatable chamber 1130, or in a position closer to the superior end 1112 relative to the first inflatable chamber 1130.

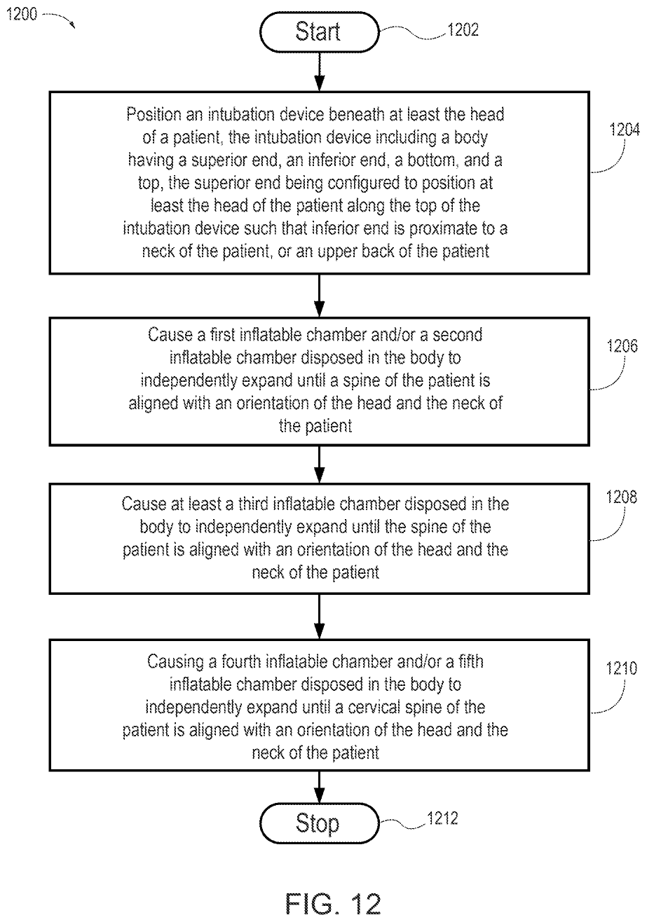

Referring now to FIG. 12, a diagram 1200 illustrates an example technique for positioning a patient using an intubation positioning device, such as a patient that may be lying on a surgical table. At 1202, the process may start or restart. At 1204, an intubation device may be positioned beneath at least the head of the patient lying on the surgical table. The intubation device may include a body having a superior end, an inferior end, a bottom, and a top. The superior end may be configured to position at least the head of the patient along the top of the intubation device such that inferior end may be proximate to a neck of the patient, and/or an upper back of the patient.

At 1206, a first inflatable chamber and/or a second inflatable chamber disposed in the body may be caused to independently expand until a spine of the patient may be aligned with an orientation of the head and the neck of the patient.

At 1208, at least a third inflatable chamber disposed in the body may be caused to independently expand until the spine of the patient is aligned with an orientation of the head and the neck of the patient. At 1210, a fourth inflatable chamber or a fifth inflatable chamber disposed in the body may be caused to independently expand until a cervical spine of the patient may be aligned with an orientation of the head and the neck of the patient. Not shown, at least a sixth inflatable chamber disposed in the body may be caused to independently expand until a thoracic spine of the patient may be aligned with an orientation of the head and the neck of the patient. At 1212, the process may stop or restart.

Those skilled in the art will appreciate that the disclosed subject matter improves upon methods and/or apparatuses for intubation by providing devices and/or techniques for controlling an alignment of an intubation patient's head, neck (e.g., cervical spine), and/or upper back (e.g., thoracic spine) into a desired/optimum orientation for successful intubation.

The subject matter of this disclosure, and components thereof, can be realized by instructions that upon execution cause one or more processing devices to carry out the processes and/or functions described herein. Such instructions can, for example, comprise interpreted instructions, such as script instructions, e.g., JavaScript or ECMAScript instructions, or executable code, and/or other instructions stored in a computer readable medium.

Implementations of the subject matter and/or the functional operations described in this specification and/or the accompanying figures can be provided in digital electronic circuitry, in computer software, firmware, and/or hardware, including the structures disclosed in this specification and their structural equivalents, and/or in combinations of one or more of them. The subject matter described in this specification can be implemented as one or more computer program products, e.g., one or more modules of computer program instructions encoded on a tangible program carrier for execution by, and/or to control the operation of, data processing apparatus.

A computer program (also known as a program, software, software application, script, or code) can be written in any form of programming language, including compiled or interpreted languages, and/or declarative or procedural languages. It can be deployed in any form, including as a stand-alone program or as a module, component, subroutine, and/or other unit suitable for use in a computing environment. A computer program may or might not correspond to a file in a file system. A program can be stored in a portion of a file that holds other programs and/or data (e.g., one or more scripts stored in a markup language document), in a single file dedicated to the program in question, and/or in multiple coordinated files (e.g., files that store one or more modules, sub programs, or portions of code). A computer program can be deployed to be executed on one computer or on multiple computers that may be located at one site or distributed across multiple sites and/or interconnected by a communication network.

The processes and/or logic flows described in this specification and/or in the accompanying figures may be performed by one or more programmable processors executing one or more computer programs to perform functions by operating on input data and/or generating output, thereby tying the process to a particular machine (e.g., a machine programmed to perform the processes described herein). The processes and/or logic flows can also be performed by, and apparatus can also be implemented as, special purpose logic circuitry, e.g., an FPGA (field programmable gate array) and/or an ASIC (application specific integrated circuit).

Computer readable media suitable for storing computer program instructions and/or data may include all forms of non-volatile memory, media and memory devices, including by way of example semiconductor memory devices (e.g., EPROM, EEPROM, and/or flash memory devices); magnetic disks (e.g., internal hard disks or removable disks); magneto optical disks; and/or CD ROM and DVD ROM disks. The processor and/or the memory can be supplemented by, or incorporated in, special purpose logic circuitry.

While this specification and the accompanying figures contain many specific implementation details, these should not be construed as limitations on the scope of any invention and/or of what may be claimed, but as descriptions of features that may be specific to described example implementations. Certain features that are described in this specification in the context of separate implementations can also be implemented in combination in perhaps one implementation. Various features that are described in the context of perhaps one implementation can also be implemented in multiple combinations separately or in any suitable sub-combination. Although features may be described above as acting in certain combinations and/or perhaps even (e.g., initially) claimed as such, one or more features from a claimed combination can in some cases be excised from the combination. The claimed combination may be directed to a sub-combination and/or variation of a sub-combination.

While operations may be depicted in the drawings in an order, this should not be understood as requiring that such operations be performed in the particular order shown and/or in sequential order, and/or that all illustrated operations be performed, to achieve useful outcomes. The described program components and/or systems can generally be integrated together in a single software product and/or packaged into multiple software products.

Examples of the subject matter described in this specification have been described. The actions recited in the claims can be performed in a different order and still achieve useful outcomes, unless expressly noted otherwise. For example, the processes depicted in the accompanying figures do not require the particular order shown, and/or sequential order, to achieve useful outcomes. Multitasking and parallel processing may be advantageous in one or more embodiments.

While the present disclosure has been illustrated and described in detail in the drawings and foregoing description, the same is to be considered as illustrative and not restrictive in character, it being understood that only certain examples have been shown and described, and that all changes and modifications that come within the spirit of the present disclosure are desired to be protected.

What is claimed is:

1. An intubation positioning device configured to selectively adjust a head, neck, and upper back of a patient lying on a surgical table, the intubation device comprising:
a body having a top, a bottom, a superior end relative to the neck, an inferior end relative to the neck, the inferior end spaced from the superior end along a longitudinal direction, and at least two lateral sides that extend from the superior end to the inferior end along the longitudinal direction, the body including an outer casing that permits expansion and contraction of the body in at least a vertical direction that is transverse to the longitudinal direction, wherein at least a part of the superior end is at least one of: sized to receive the head of the patient, or shaped to receive the head of the patient;
a first inflatable chamber disposed in the body and substantially oriented along the longitudinal direction;
a second inflatable chamber disposed in the body and substantially oriented along the longitudinal direction, the second inflatable chamber disposed in a contiguous position relative to the first inflatable chamber;
a first inflation port coupled to the first inflatable chamber, the first inflation port configured to permit a fluid to enter the first inflatable chamber;
a second inflation port coupled to the second inflatable chamber, the second inflation port configured to permit the fluid to enter the second inflatable chamber; and
a controller coupled to the first and second inflation ports, wherein the controller is configured to: selectively permit entrance of the fluid into one or more of the first inflatable chamber and the second inflatable chamber, and selectively transition the first inflatable chamber and the second inflatable chamber, independently, between an inflated shape and a deflated shape.

2. The device of claim 1, further comprising:
a third inflatable chamber disposed in the body and substantially oriented along the longitudinal direction, the third inflatable chamber disposed in a contiguous position relative to the second inflatable chamber; and
a third inflation port coupled to the third inflatable chamber, the third inflation port configured to permit the fluid to enter the third inflatable chamber, the controller being coupled to the third inflation port, wherein the controller is further configured to: selectively permit entrance of the fluid into the third inflatable chamber, selectively transition the third inflatable chamber, independently from the first inflatable chamber and the second inflatable chamber, between an inflated shape and a deflated shape.

3. The device of claim 1, wherein the first inflatable chamber and the second inflatable chamber are configured to independently transition between a fully-deflated shape, a partially inflated shape, and a fully-inflated shape.

4. The device of claim 3, wherein the superior end, the inferior end, and the at least two lateral sides extend substantially perpendicular to the top and the bottom as at least one of: the first inflatable chamber or the second inflatable chamber is transitioned from the fully-deflated shape to the fully-inflated shape.

5. The device of claim 1, where the outer casing of the body is constructed of a flexible material.

6. The device of claim 1, wherein the first inflation port and the second inflation port are at least one of: a valve, a gauge cock, a plug, a tap, or a spigot.

7. The device of claim 2, further comprising:
a fourth inflatable chamber disposed in the body and substantially oriented along the longitudinal direction, the fourth inflatable chamber disposed in a contiguous position relative to the second inflatable chamber;
a fifth inflatable chamber disposed in the body and substantially oriented along the longitudinal direction, the fifth inflatable chamber disposed in a contiguous position relative to the fourth inflatable chamber; and
a sixth inflatable chamber disposed in the body and substantially oriented along the longitudinal direction, the sixth inflatable chamber disposed in a contiguous position relative to the fourth inflatable chamber.

8. The device of claim 7, further comprising:
a fourth inflation port coupled to the fourth inflatable chamber, the fourth inflation port configured to permit the fluid to enter the fourth inflatable chamber, the controller being coupled to the fourth inflation port, wherein the controller is further configured to: selectively permit entrance of the fluid into the fourth inflatable chamber, and selectively transition the fourth inflatable chamber, independently from the first inflatable chamber, the second inflatable chamber, the third inflatable chamber, the fifth inflatable chamber, and the sixth inflatable chamber, between an inflated shape and a deflated shape.

9. The device of claim 7, further comprising:
a fifth inflation port coupled to the fifth inflatable chamber, the fifth inflation port configured to permit the fluid to enter the fifth inflatable chamber, the controller being coupled to the fifth inflation port, wherein the controller is further configured to: selectively permit entrance of the fluid into the fifth inflatable chamber, and selectively transition the fifth inflatable chamber, independently from the first inflatable chamber, the second inflatable chamber, the third inflatable chamber, the fourth inflatable chamber, and the sixth inflatable chamber, between an inflated shape and a deflated shape.

10. The device of claim 7, further comprising:
a sixth inflation port coupled to the sixth inflatable chamber, the sixth inflation port configured to permit the fluid to enter the sixth inflatable chamber, the controller being coupled to the sixth inflation port, wherein the controller is further configured to: selectively permit entrance of the fluid into the sixth inflatable chamber, and selectively transition the sixth inflatable chamber, independently from the first inflatable chamber, the second inflatable chamber, the third inflatable chamber, the fourth inflatable chamber, and the fifth inflatable chamber, between an inflated shape and a deflated shape.

11. The device of claim 7, wherein the third inflatable chamber, the fourth inflatable chamber, the fifth inflatable chamber, and the sixth inflatable chamber are configured to independently transition between a fully-deflated shape, a partially inflated shape, and a fully-inflated shape.

12. The device of claim 1, wherein the first inflatable chamber and the second inflatable chamber define at least a portion of the outer casing.

13. The device of claim 1, wherein the body has a length that extends along the longitudinal direction from the superior end to the inferior end, a width that extends from a first lateral side of the at least two lateral sides to a second lateral side of the at least two lateral sides along a transverse direction that is perpendicular to the longitudinal direction and the vertical direction, wherein the length is greater than the width when the body is positioned on the surgical table in preparation to receive the patient.

14. The device of claim 1, wherein the body has a length that extends along the longitudinal direction from the superior end to the inferior end, a width that extends from a first lateral side of the at least two lateral sides to a second lateral side of the at least two lateral sides along a transverse direction that is perpendicular to the longitudinal direction and the vertical direction, wherein the width is greater than the length when the body is positioned on the surgical table in preparation to receive the patient.

15. The device of claim 1, wherein the superior end defines a first height that extends from the bottom to the top along the vertical direction, and the inferior end defines a second height that extends from the bottom to the top along a vertical direction, the first height being greater than the second height upon at least a partial inflation of at least one of: the first inflatable chamber or the second inflatable chamber.

16. The device of claim 7, wherein the body is configured such that:
the first inflatable chamber is disposed substantially contiguous to the second inflatable chamber in at least one of: a position above the second inflatable chamber relative to the vertical direction, or a position beneath the second inflatable chamber relative to the vertical direction, the first inflatable chamber being disposed closer to the superior end relative to the third inflatable chamber;
the third inflatable chamber is disposed substantially contiguous to the fourth inflatable chamber in at least one of: a position above the fourth inflatable chamber relative to the vertical direction, or a position beneath the fourth inflatable chamber relative to the vertical direction;
the fourth inflatable chamber is disposed relative to the second inflatable chamber in a least one of: a substantially lateral contiguous position, or a substantially diagonal contiguous position;
the fifth inflatable chamber is disposed substantially contiguous to the sixth inflatable chamber in at least one of: a position above the sixth inflatable chamber relative to the vertical direction, or a position beneath the sixth inflatable chamber relative to the vertical direction; and
the sixth inflatable chamber is disposed relative to the third inflatable chamber in a least one of: a substantially lateral contiguous position, or a substantially diagonal contiguous position, the sixth inflatable chamber being disposed closer to the inferior end relative to the third inflatable chamber.

17. The device of claim 7, wherein the body is configured such that:
the first inflatable chamber is disposed in a substantially lateral contiguous position relative to at least one of: the second inflatable chamber, the fourth inflatable chamber, or the fifth inflatable chamber, the first inflatable chamber being disposed closer to the superior end relative to the fourth inflatable chamber;
the second inflatable chamber is disposed in a substantially lateral contiguous position relative to at least one of: the first inflatable chamber, the third inflatable chamber, the fourth inflatable chamber, or the fifth inflatable chamber;
the fifth inflatable chamber is disposed in a substantially lateral contiguous position relative to at least one of: the second inflatable chamber, the third inflatable chamber, the fourth inflatable chamber, or the sixth inflatable chamber; and
the sixth inflatable chamber is disposed in a substantially lateral contiguous position relative to at least one of: the fourth inflatable chamber, or the fifth inflatable chamber, the sixth inflatable chamber being disposed closer to the inferior end relative to the fourth inflatable chamber.

18. The device of claim 2, wherein the body is configured such that:
the first inflatable chamber is disposed substantially contiguous to the second inflatable chamber in at least one of: a position above the second inflatable chamber relative to the vertical direction, or a position beneath the second inflatable chamber relative to the vertical direction, the first inflatable chamber being disposed closer to the superior end relative to the third inflatable chamber; and
the third inflatable chamber is disposed substantially contiguous to at least one of: the first inflatable chamber, or the second inflatable chamber, the third inflatable chamber being disposed closer to the inferior end relative to the first inflatable chamber.

19. The device of claim 2, wherein the body is configured such that:
the first inflatable chamber is disposed in a substantially lateral contiguous position relative to the second inflatable chamber; and
the third inflatable chamber is disposed substantially contiguous to at least one of: the first inflatable chamber, or the second inflatable chamber, the third inflatable chamber being disposed closer to the inferior end relative to the first inflatable chamber.

20. The device of claim 1, wherein the body is configured such that:
the first inflatable chamber is disposed substantially contiguous to the second inflatable chamber in at least one of: a position above the second inflatable chamber relative to the vertical direction, or a position beneath the second inflatable chamber relative to the vertical direction; and
the first inflatable chamber being disposed in at least one of: a position closer to the bottom relative to the second inflatable chamber, or a position closer to the top relative to the second inflatable chamber.

21. The device of claim 1, wherein the body is configured such that:
the first inflatable chamber is disposed in a substantially lateral contiguous position relative to the second inflatable chamber; and
the second inflatable chamber being disposed in at least one of: a position closer to the inferior end relative to the first inflatable chamber, or a position closer to the superior end relative to the first inflatable chamber.

22. A method for positioning at least a head of a patient lying on a surgical table, the method comprising:
positioning an intubation device beneath at least the head of the patient lying on the surgical table, the intubation device including a body having a superior end, an inferior end, a bottom, and a top, wherein the superior end is configured to position at least the head of the patient along the top of the intubation device such that inferior end is proximate to at least one of: a neck of the patient, or an upper back of the patient; and
causing one or more of: a first inflatable chamber or a second inflatable chamber disposed in the body to independently expand until a spine of the patient is aligned with an orientation of the head and the neck of the patient.

23. The method of claim 22, wherein the causing further comprises causing at least a third inflatable chamber disposed in the body to independently expand until the spine of the patient is aligned with an orientation of the head and the neck of the patient.

24. The method of claim 22, wherein the causing further comprises causing one or more of: a fourth inflatable chamber or a fifth inflatable chamber disposed in the body to independently expand until a cervical spine of the patient is aligned with an orientation of the head and the neck of the patient.

25. The method of claim 22, wherein the causing further comprises causing at least a sixth inflatable chamber disposed in the body to independently expand until a thoracic spine of the patient is aligned with an orientation of the head and the neck of the patient.
